(19) United States
(12) Patent Application Publication
(10) Pub. No.: US 2001/0009764 A1
(43) Pub. Date: Jul. 26, 2001

(54) Endothelial-derived toxic factor

(76) Inventors: Paula Grammas, Edmond, OK (US); Paul H. Weigel, Edmond, OK (US)

Correspondence Address: Dunlap, Codding & Rogers, P.C., Attn: Kathryn L. Hester, Suite 420, 9400 North Broadway, Oklahoma City, OK, 73114, US

(21) Appl. No.: 09/733,399

(22) Filed: Dec. 8, 2000

Related U.S. Application Data
(62) Division of application No. 09/189,269, filed on Nov. 10, 1998.
(60) Provisional application No. 60/065,784, filed on Nov. 11, 1997.

(51) Int. Cl. A61K 39/395; C07K 1/00; C07K 14/00; C07K 17/00

(52) U.S. Cl. 435/7.1; 435/7.2; 530/350; 424/152.1

(57) ABSTRACT

A heat-labile, trypsin-sensitive protein of MW 10-50 kDa which is produced by microvessels from patients suffering from Alzheimer's disease or which is derived from mammalian vascular endothelial cells treated to inhibit protein kinase C. The protein is specifically toxic to neuronal cells and is called endothelial-derived toxic factor (EDTF). EDTF acts by inducing necrosis or apoptosis of neuronal cells. Hybridomas which secrete monoclonal antibodies have been raised against EDTF. The antibodies can be used in therapies or in diagnostic assays to detect the presence of EDTF in a body fluid. EDTF can be used in screening assays to identify compounds which inhibit synthesis or activity of EDTF.

### CROSS-REFERENCE TO RELATED APPLICATIONS

The present application is a divisional of U.S. Ser. No. 09/189,269, filed Nov. 10, 1998, entitled: “ENDOTHELIAL-DERIVED TOXIC FACTOR”, which claims the benefit of U.S. Provisional Application Serial No. 60/065,784, filed Nov. 11, 1997, which is hereby incorporated by reference herein in its entirety.

### STATEMENT REGARDING FEDERALLY SPONSORED RESEARCH OR DEVELOPMENT

This work was supported in part by NIH grant NS30457 (PG). The U.S. Government may have certain rights to this invention.

### BACKGROUND

Alzheimer's disease (AD) is a neurodegenerative disorder affecting 4 million older Americans. Despite intense investigation, the cause remains unknown. AD is a dementia characterized by amyloid β(Aβ) deposition, plaques, tangles and neuronal cell loss. The familial forms of early-onset AD, 2-7% of AD patients, are associated with genes for presenilin proteins located on chromosomes 1 and 14, and the β amyloid precursor protein (βAPP) on chromosome 21. Inheritance of the ApoE allele ε4 (chromosome 19) is a risk factor for the development of late-onset AD. The development of a transgenic mouse model that over-expresses a mutated form of βAPP695 is significant, because this animal demonstrates not only Aβ elevation and amyloid plaques, but also memory impairment. While Aβ is a key factor in the pathology of the AD brain, it alone can not account for the neuronal cell death that underlies AD dementia. Indeed, despite intensive research efforts, the causes of neuronal loss, the most common (sporadic) form of AD, are unknown. Recent work showing antibody reactivity to a “new” 100 kDa protein throughout the AD brain is exciting, and supports the notion that heretofore unknown factors likely play a role in the pathogenesis of AD.

An important role for blood vessels in the pathogenesis of AD dementia has been supported by recent studies. Snowdon and colleagues documented cortex plaque and tangle pathology consistent with diagnosis of AD without dementia or evidence of synapse loss. Dementia was only evident in patients with this AD pathology and cerebrovascular disease (i.e., brain infarcts). They concluded that cerebrovascular disease plays a role in determining the presence and severity of dementia. In addition, a 15 year longitudinal study of blood pressure and dementia also supported the concept that vascular factors are involved in the development of AD. A recent population-based cross-sectional study has linked atherosclerosis, the ApoE genotype, and prevalence of dementia. Finally, ApoE4, a major risk factor for AD, is also a significant factor in development of cerebral amyloid angiopathy, an important feature of most AD cases.

In previous studies, numerous structural and functional cerebromicrovascular abnormalities in AD have been identified. Decreased microvascular density and vascular distortions such as vessel kinking, twisting, tortuosity and looping occur in AD. It has been suggested that neuronal loss in AD may result from pathologic changes in vessel angioarchitecture, decreased cerebral blood flow, and altered oxygen utilization leading to cerebral “microcirculatory impairment”. In addition, active functions of the blood-brain barrier, including glucose transport, are diminished in AD. We have previously demonstrated that AD brain microvessels in vitro show receptor changes, signaling defects, especially in protein kinase C and cAMP pathways, and overproduction of nitric oxide. Elevated vascular production of nitric oxide, a potentially neurotoxic mediator in the brain, may contribute to neuronal injury and death in AD. These data taken together suggest that vessels are dysfunctional in AD. The cerebral circulation as a target of injury in AD is likely because the brain endothelial cell is the only cell type in the CNS that is continuously exposed to potentially noxious elements and inflammatory mediators present in the blood.

One of the most important and yet overlooked aspects of the etiology and pathogenesis of AD is that it is an age-related condition. Indeed, age-related penetrance is such that even in predisposed individuals, disease onset rarely occurs before age 55. There is little information as to vascular function in the aged brain. A change in smooth muscle reactivity and a significant increase in choline acetyltransferase in the cerebral arteries of 22 month old Fischer 344 rats have been shown. Abnormalities have been documented in choline and peptide transport in the rat cerebral microcirculation in aging, suggesting an aging effect on active processes at the blood-brain barrier. Age-related changes in cerebral microvessel membrane fluidity and protein and lipid composition have also been reported, and are likely to be important for receptor/effector coupling and the efficiency of signal transduction cascades in the aged brain.

Microvascular pathology has been found to occur in both AD and aged vessels but is quantitatively more advanced in dementia. Similarly, structural changes in cerebral capillaries in elderly patients correlate positively with advanced age and dementia. Recently workers showed that increased amounts of soluble Aβ were found in the vessels compared to controls. Interestingly, microvessels from aged rats also showed an increased release of the carboxyl-fragment of βAPP compared to controls, again suggesting a baseline level of vascular dysfunction in aging. Thus, the vascular pathology and dysfunction that occurs in AD may be superimposed on vascular injury present as a consequence of aging. In other words, the cumulative effects of aging and the presence of AD could produce a critical loss of neurons leading to clinical impairment of mental function.

Evidence that nonsteroidal anti-inflammatory drugs exert beneficial effects in AD supports the concept that inflammation plays a significant role in this disorder. The inflammatory response in the Alzheimer's brain is characterized by the presence of activated microglia, expressing interleukin (IL)-1 and IL-6 and class II major histocompatibility antigens. Amyloid deposition is associated with inflammatory cytokines and reactive glial cells. Furthermore, complement (Clq, C4, C3) and acute phase proteins (α₁-antichymotrypsin, α₂-macroglobulin) are also associated with diffuse and classical neuritic plaques. Data from one of our laboratories indicate that CAP37, a multi-functional protein isolated from the granules of human neutrophils, is elevated in AD brains. CAP37 plays a role in inflammation, is chemotactic for monocytes, and can stimulate monocyte adhesion to endothelial cells. A monospecific antiserum to CAP37 reacts with blood vessels in AD brains but not in controls, implicating this novel inflammatory mediator in this disorder.

Endothelial cells are key cellular regulators of inflammatory responses and are an important source of cytokines such as IL-1, IL-6, and IL-8. The relevance of these soluble mediators in AD is suggested by experiments showing an increase in IL-6 in aging and an elevation of both IL-1 and IL-6 in AD brains. IL-1 induces generation of nitric oxide and IL-6 overexpression in the CNS of transgenic mice is associated with a range of structural and functional impairments. The notion that damaged or abnormal endothelial cells contribute inflammatory mediators to the AD disease process is supported by our data showing that “injury” of brain endothelial cells in culture by Aβ evokes the expression of CAP37 in these cells. Finally, evidence that inflammation of endothelial cells occurs in AD is supported by the demonstration that intercellular adhesion molecule-1 (ICAM-1), a surface glycoprotein found on activated endothelial cells, is expressed in AD lesions. These data are consistent with a large body of literature documenting that endothelial cells respond to injury with alterations in mediator generation and surface molecule expression.

Considerable interest has focused on the pathways that mediate cell death in the nervous system. Necrosis and apoptosis are two distinct mechanisms of cell death, differing in their effects on cellular morphology and metabolism. Necrosis is usually evoked by intense insults and is characterized by cell swelling, membrane lysis, injury to cytoplasmic organelles, and release of cellular contents. Apoptosis is an active cellular process that can be triggered by both receptor and nonreceptor-mediated signaling pathways. The apoptotic cell death program is defined by cell shrinkage, membrane blebbing, nuclear pyknosis, chromatin condensation, and genomic fragmentation. It is also now appreciated that RNA and protein synthesis are not always required and that the apoptotic cascade can be activated directly, because most cells express inactive, but potentially lethal, proteins. While necrosis and apoptosis are distinct entities, they may also represent extremes of a cell death continuum. In this regard, the same stimulus can evoke both apoptosis or necrosis, depending on the intensity and duration of the stimulus as well as the status of the target cell. For example, cerebral cortical cultures were shown to undergo both apoptosis and necrosis in response to N-methyl-D-aspartate and nitric oxide.

The apoptotic process may be qualitatively different in neurons than in other cell types that have been examined, since neurons are post-mitotic cells. However, molecules unique to neuronal apoptosis, have not been identified. Phosphoprotein p53 (p53), c-Jun amino-terminal kinase (JNK), interleukin converting enzymes (ICE), phosphatidylinositol 3-kinase (PI 3-K), protein kinase B/Akt (PKB), have all been reported to play important roles in the balance between neuronal survival and apoptosis (see below) and thus could be targets for the neurospecific toxin described herein.

### SUMMARY OF THE INVENTION

The present invention contemplates a heat-labile, trypsin-sensitive type of protein of MW 10-50 kDa which is produced by microvessels from patients suffering from Alzheimer's disease or from other diseases, or which is derived from mammalian vascular endothelial cells treated to inhibit protein kinase C. The protein, called endothelial-derived toxic factor (EDTF), is specifically toxic to neuronal cells and can act via necrosis or apoptosis. Hybridomas which secrete monoclonal antibodies have been raised against EDTF. The present invention contemplates a method for detecting the presence of endothelial derived toxic factor in a sample, comprising incubating said sample with a monoclonal antibody which possesses high affinity binding for endothelial-derived toxic factor under conditions which provide for the formation of an endothelial-derived toxic factor-antibody complex; and detecting the presence of said endothelial-derived toxic factor-antibody complex to determine whether endothelial-derived toxic factor is present in the sample.

The present invention further contemplates a method of screening for compounds which inhibit the necrosis or apoptosis-inducing effects of EDTF on neuronal cells comprising providing a sample of neuronal cells; treating the sample with a test compound; exposing the treated sample to EDTF; and examining the neuronal cells for evidence of inhibition or reduction of apoptosis or necrosis of the neuronal cells.

The present invention contemplates a further embodiment comprising a method of screening for compounds which inhibit expression or activation of EDTF in microvascular endothelial cells comprising providing a sample of microvascular endothelial cells; treating the sample with a test compound; exposing the treated sample to a condition which normally induces the production of EDTF; and examining the exposed sample for production of EDTF.

The monoclonal antibodies of the present invention may be used therapeutically to treat a subject suffering from Alzheimer's disease or from a condition having as a symptom an excessive production of EDTF.

### DESCRIPTION OF THE DRAWINGS

FIGS. 1A-1D. Conditioned medium from AD microvessels causes neuronal cell death. Conditioned medium was prepared under standardized conditions (100 μg protein/ml) by incubating brain microvessels from AD (73±5.7 yr, range 62-80 yr), age-matched non-AD (71.8±5.7 yr, range 59-81 yr), and non-AD young patients 43.9±13.5 yr, range 21-58 yr) at 37° C. for 4 h. Different volumes of conditioned medium were placed into 24 well plates with primary cerebral cortical cultures, incubated at 37° C. for 24 h, and 100 μl aliquots of medium were then assayed for neuronal lactate dehydrogenase (LDH) release. Each of FIGS. 1A-1C shows 3 typical individual cases (▾, ▴, ▪) examined over the indicated volume range of microvessel-conditioned media: AD (FIG. 1A), non-AD aged (FIG. 1B), and non-AD young (FIG. 1C). For FIG. 1D the mean slopes ± standard error of the mean from a larger number of AD (n=9), non-AD aged (n=9) and non-AD young (n=6) cases were determined. The slope is defined as the neurotoxicity of the sample: =% cell death/μl conditioned medium. Slopes were determined by linear regression analysis, and had an average correlation coefficient (n=24) of 0.96.

*p<0.001, significantly different from non-AD young;

**p<0.001, significantly different from non-AD aged;

***p<0.001, significantly different from AD.

FIG. 2. AD microvessel-conditioned medium evokes neuronal cell death comparable to AD microvessels in co-culture. Conditioned medium from AD microvessels was collected at 1, 4, 9, and 24 h. Primary cerebral cortical cultures were treated with AD microvessels (100 μg) in co-culture or conditioned medium (1 ml) and incubated 4 h at 37° C. Aliquots of the culture medium (100 μl) were then removed and neuronal cell death was assessed by LDH release. Data are means ± SEM of 2 separate experiments each performed in triplicate.

FIG. 3. Dose-effect of MV protein on neurotoxicity. Variable amounts of MV protein from (10-200 μg/well) were added to the culture dish insert. Co-cultures were incubated for 4 or 24 hrs, and 100 μl aliquots were assayed for LDH. Each bar represents the mean of 3 separate experiments performed in duplicate. Data are expressed as a percentage of total LDH release.

FIG. 4. AD microvessel cytotoxicity is neurospecific. MVs from AD brains were seeded onto Millipore filter inserts and co-cultured in 24 well plates with several cell types including: primary rat cerebellar granule neurons (CGN), primary rat cerebral cortical cultures (CCC), rat brain glial cells (GLIA), rat brain endothelial cells (RBEC), rat fibroblasts (FIB), rat aorta smooth muscle cells (RASMC), rat brain smooth muscle cells (RBSMC), bovine aorta endothelial cells (BAEC), and bovine retinal endothelial cells (BREC). After 4 hrs, 100 μl aliquots of media were assayed for LDH release. Cell death (% cytotoxicity) was determined by release of LDH into the media. Inset: Undifferentiated PC-12 cells (PC-12-U) and PC-12 cells which had been differentiated by treatment with 50 ng/ml nerve growth factor (NGF) for 7 days (PC-12-D) were used with MVs as above. Each bar represents the mean ± SEM of 3 experiments performed in duplicate. Data are expressed as a percentage of total LDH release.

FIG. 5. The vascular-derived neurotoxic factor is trypsin-sensitive and heat-labile. Microvessel-conditioned medium was collected and divided into 4 samples that were subjected to either no treatment (CM), heat (55° C. for 40 min), trypsin (0.1 mg/ml TPCK-treated trypsin for 1 h at 37° C. followed by soy bean trypsin inhibitor (SBTI) at 1.0 mg/ml), or trypsin and SBTI added together before addition to the conditioned medium. These conditioned media were then added to primary cerebral cortical cultures and neuronal cell death was assessed by LDH release after 24 h. Data are means ± SEM of 3 separate experiments each performed in triplicate.

FIG. 6. The neurotoxic factor produced by AD microvessels evokes either apoptosis or necrosis. Conditioned media (collected after 4 h) from variable amounts of AD microvessels (25-200 μg protein) were added to primary cerebral cortical cultures. At 4 h, an aliquot of culture medium was removed and assayed for LDH release ( - - - ). At 24 h, the same cells were then solubilized and apoptosis (—) was determined by ELISA measurement of nucleosomes as described below. Each point represents the average of duplicates.

FIGS. 7A-7C. Neuronal apoptosis induced by AD microvessels is time and dose-dependent. A. Varying amounts of AD microvessels were co-cultured with cerebral cortical cultures for 24 h and apoptosis was quantified by ELISA, as described for FIG. 6. B and C. After various times (1-24 h) of medium conditioning by AD microvessels, the conditioned medium was centrifuged and 10 μl (B) or 50 μl (C) was added to cerebral cortical cultures, which were then incubated for 24 h prior to performing the ELISA nucleosome assay for apoptosis. Each bar represents the mean ± SEM of 2 experiments performed in duplicate.

FIG. 8. Specificity of endothelial derived toxic factor EDTF response to Protein Kinase C inhibition. To confirm that production of EDTF in response to BIM (EDTF^{BIM}) was indeed related to PKC inhibition, the effect of other PKC inhibitors, calphostin (Cal) and stauroporine (S'sporin) as well as the PKC agonist PMA, were examined. Endothelial cell cultures were incubated in serum-free media (control) containing either BIM (1 μM); BIM and PMA (1 μM each); calphostin (1 μM); or staurosporine (1 μM). The media were collected after 24 hours and added to neuronal cultures and cytotoxicity (LDH release) was measured after 24 hours. Data are expressed as a percentage of the BIM-mediated cytotoxicity (100%).

FIG. 9. Time Course of EDTF Appearance. To determine the kinetics of mediator appearance, endothelial cells were treated with BIM (1 μM) in serum-free DMEM and media samples were collected at timed intervals. The results demonstrate that the mediator was not present before 6-8 hours. EDTF production by endothelial cells may therefore require gene expression.

FIGS. 10A-10B. EDTF size characterization. Conditioned media containing EDTF^{BIM} were sequentially placed into 50- and 10-kDa CENTRICON™ devices. Fractions (top and bottom) from both devices were collected and added to neuronal cell cultures, and cytotoxicity was measured (FIG. 10A). EDTF produced by microvessels (MV) in culture (EDTF^{MV}) were sequentially placed into 100-, 50-, and 10-kDa CENTRICON™ devices. Bottom fractions were collected (FIG. 10B). The cytotoxicity (LDH release) is expressed as a percentage of the total LDH released after treatment with Triton X-100.

FIG. 11 shows the degrees of effectiveness (immunoprecipitation) of five monoclonal antibodies against EDTF^{BIM}. The toxicity mediated by EDTF alone is 100%. Monoclonal antibodies from hybridoma culture supernatants (clones designated on x-axis) were immobilized on protein A Sepharose that had been pre-loaded with rabbit antibodies against mouse immunoglobulin and incubated with an equal volume of EDTF at 4° C. for 4 h with gentle agitation. The complexes were centrifuged, media diluted 1:4 with serum-free media containing lactalbumin hydrolysate and added to neuronal cultures. Neuronal cell death (LDH) release was determined after 24 h.

FIG. 12. Effect of monoclonal antibodies on neurotoxicity of conditioned culture media. Monoclonal antibody from hybridoma lines 2e4F was isolated from ascites fluid by protein G affinity chromatography, eluted and immobilized again by binding to protein A Sepharose. The antibody-protein A sepharose conjugates were then added to supernatants from cultures of Alzheimer's disease patients' brain microvessels (mv) or rat brain endothelial cells that had been cultured in serum free medium containing the protein kinase C inhibitor bis-indolylmaleimide (cm+). After overnight incubation, the antibody-protein A sepharose conjugates were removed by centrifugation and the treated culture supernatants were added to cultured rat brain neurons to assay for neurotoxic activity. After 24 hours, lactate dehydrogenase (LDH) activity in the medium and total lactate dehydrogenase activity, released after lysing cells with detergent, were determined. Neurotoxicity is expressed as percent lactate dehydrogenase activity released prior to cell lysis. The background value for the cm− samples (≈5%) has been subtracted.

### DESCRIPTION OF THE INVENTION

It is demonstrated herein that blood microvessels isolated from the brains of AD patients, in co-culture with neurons, evoke neuronal cell death by both apoptotic and necrotic mechanisms by secreting a toxic vascular-derived protein referred to herein as endothelial-derived toxic factor (specifically, EDTF^{MV}). In contrast, young healthy brain blood vessels do not produce this lethal factor. Furthermore, this factor is neurospecific, eliciting death in primary neuronal cultures and in the differentiated PC-12 neuronal cell line, but not in nonneuronal cells. These novel findings indicate that in patients suffering from AD, brain microvessels can produce a soluble protein factor (EDTF^{MV}) which injures or kills neurons.

While the clinical presentation (dementia) and neuropathology (Aβ deposition, neuritic plaques and neurofibrillary tangles) of AD are uniform, the pathogenesis of this disorder is likely to be multifactorial. Our findings are the first to identify the vasculature as a source of neurotoxic molecules. Our experiments indicate that vessels from aged patients (65 yrs) evoke lethal injury in cultured neurons, albeit less than that of AD vessels, whereas no toxicity is demonstrable using adult-derived (30-60 yrs) vessels.

### Results Using Human Brain Microvessels

A unique in vitro model system was established to directly investigate the role human brain microvessels (MV) have in producing soluble protein factors which may play a role in the injury or killing of neurons.

### Establishment of in vitro System

Human Microvessel Isolation.

Microvessels were isolated from human brains using our previously published methods (Grammas P, Roher A E, and Ball M J, “Decreased α-adrenergic Receptors at the Blood-Brain Barrier in Alzheimer's Disease”, Alzheimer's Disease: Basic Mechanisms, Diagnosis and Therapeutic Strategies, K. Iqbal, D. R. C. Mclachlan, B. Winblad, H. M. Wisnieski, eds, John Wiley & Sons, Ltd., pp. 129-136, 1991; which is hereby incorporated herein by reference). Human autopsy brain specimens are obtained approximately 6-11 hrs post-mortem and frozen at −70° C. until dissection. The clinical diagnosis of primary AD is confirmed by neuropathological examination. Control samples are taken from patients who are without evidence of significant neuropathology. Isolation of cerebral MVs from human brain utilizes pooled temporal, parietal, and frontal cortices; filtration through a 210 μm sieve, collection on a 53 μm sieve, and yields 6-10 mg of microvessel protein from 15 g of human cortex. Microvessels are then resuspended in Dulbecco's modified Eagle's medium (DMEM), containing 10% fetal calf serum (FCS), and 10% dimethylsulfoxide and stored in liquid nitrogen until used.

Preparation of microvessel conditioned media:

Microvessels stored in liquid nitrogen, were quick-thawed at 37° C. and centrifuged at 2,000×g for 10 min. The vessels were washed 3 times with cold Hank's balanced salt-solution and resuspended in serum-free DMEM (2 ml) containing 1% lactalbumin hydrolysate. Microvessels were allowed to “recover” for 1 h, after which they released little LDH. The microvessels were washed by centrifugation and resuspended at a concentration of 100 μg protein per ml in serum-free DMEM containing 1% lactalbumin hydrolysate. The microvessels were then incubated for variable times (4-24 h) in a CO₂ incubator, centrifuged, and the conditioned medium was sterile-filtered through a 0.4 μm filter, and used immediately.

Primary Neuronal and PC-12 Cell Cultures.

Procedures for primary neuronal cultures prepared from fetal rat brains were previously described (Grammas P, Moore P, Weigel P H, “Production of Neurotoxic Factors by Brain Endothelium in AD”, Ann. NY Acad. Sci., 826, 1997, which is hereby incorporated herein by reference.) Neurons are obtained and cultured from 17 day fetal rats. Cerebral cortices are isolated, dissociated in Brooks-Logan solution, and seeded in polylysine-coated plates containing DMEM with 5% horse serum. After 5 days of culture, the cells are treated with 5-fluoro-deoxyuridine, and maintained for 2-3 weeks before use. The neuronal identity of cells is confirmed with an antibody to neuronal specific enolase. The purity of these cultures was determined by antibody reactivity to neuronal-specific enolase. Results of fluorescence activated cell sorting showed that approximately 88% of the cells in culture are neuronal.

PC-12 cell (rat pheochromocytoma cells; American Type Culture Collection, Rockville, Md.) cultures were maintained in RPMI 1640 media supplemented with 2 mM glutamine, 10% heat-inactivated horse serum, and 5% FCS. Cells were plated into 24 well dishes at ˜10⁵ cells/well. Seven to 14 days prior to use, 50 ng/ml nerve growth factor was added to each well to stimulate neuronal differentiation.

Determination of neuronal cell death by necrosis and apoptosis:

Cell death by necrosis was determined by release of cytoplasmic lactate dehydrogenase (LDH) (Grammas et al., “Production Of Neurotoxic Factors By Brain Endothelium In AD”, Ann NY Acad Sci, 826:47-55, 1997). Medium (100 μl) from the neuronal cultures was removed, added to a 96-well plate, the chromogenic substrate added, and the plate was incubated at room temperature for 30 min in darkness. The A₄₉₀ of each well was measured to determine LDH activity. Data (% cytotoxicity) were expressed as a percentage of total LDH released by treatment with 1% Triton X-100 added to the same well. Vascular LDH release, determined for microvessel-containing inserts placed in wells without neuronal cells, was subtracted from each co-culture point. Each point was performed in duplicate.

Cell death by apoptosis was determined by ELISA measurement of nucleosomes using a kit from Boehringer Mannheim (Indianapolis, Ind.). Neurons were collected by scraping, washed, incubated 30 min at 4° C. with lysis buffer, centrifuged at 15,000×g for 10 min at 4° C., and aliquots were transferred to a microtiter plate precoated with an antibody to histone. After washing 3 times, a second antibody to DNA, conjugated to horseradish peroxidase, was added and the plate was incubated 90 min at room temperature and washed 3 times. The peroxidase substrate was added to each well and the plate was incubated for 10-20 min. Absorbance was read at 405 nm. The amount of microvessel-mediated apoptotic neuronal cell death was expressed as a percentage of the apoptosis (100%) elicited by 10 μM sodium nitroprusside, a nitric oxide releasing compound.

Statistical analysis:

Unless otherwise indicated, data presented are means ± standard error of the mean. Statistical analysis between two groups was performed using the Student's t-test, and for comparisons among multiple samples, the analysis of variance was used.

### Results

Conditioned medium from AD microvessels causes neuronal cell death.

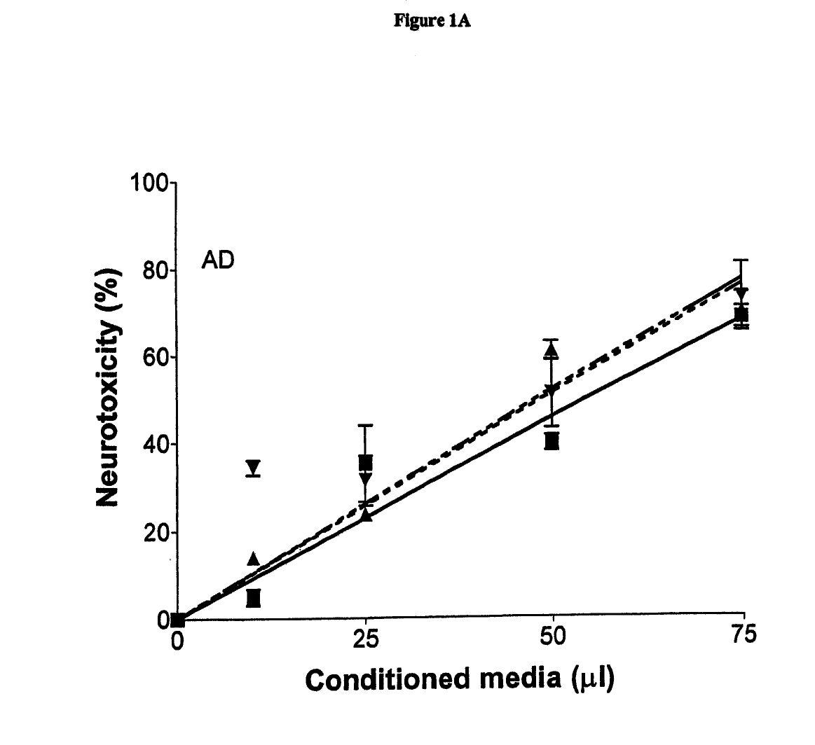

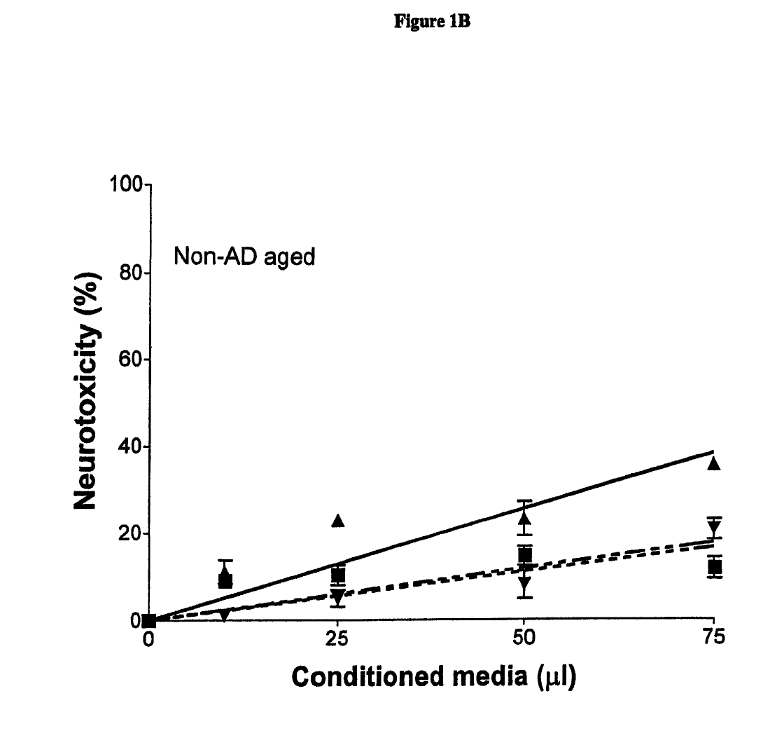

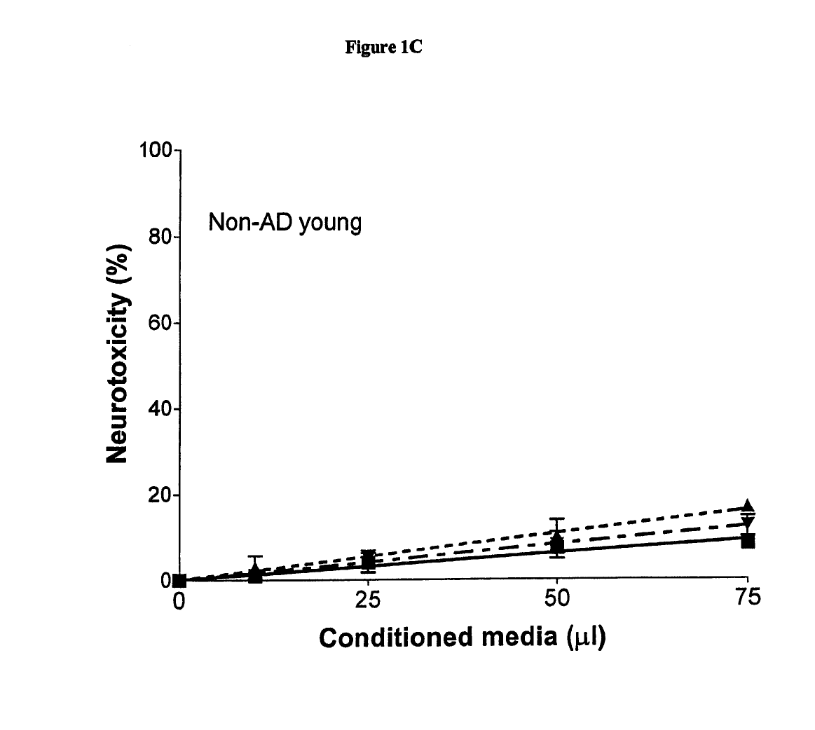

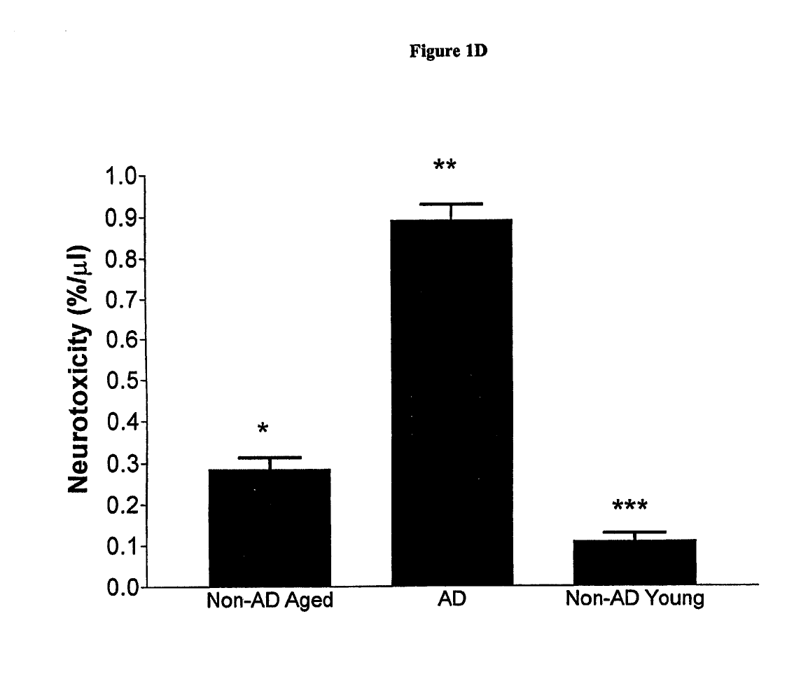

We found that either media conditioned by AD microvessels or direct co-culture of AD microvessels with neurons caused neuronal cell death. Using microvessel-conditioned media, we established a quantitative and convenient assay for neurotoxicity in which cell death was linear with the amount of conditioned medium (FIGS. 1A-1C). The neurotoxicity of conditioned media from AD brain microvessels, expressed as percent cell death per μl under standardized conditions, was significantly (p<0.001) greater than that of conditioned media from age-matched non-AD controls (FIG. 1D). The conditioned medium from microvessels of even younger (32-59 yr) non-AD patients caused essentially no neuronal death (FIG. 1C). The results are summarized in FIG. 1D for a larger number of AD and control cases.

AD microvessel-conditioned medium evokes comparable neuronal cell death to AD microvessels in co-culture.

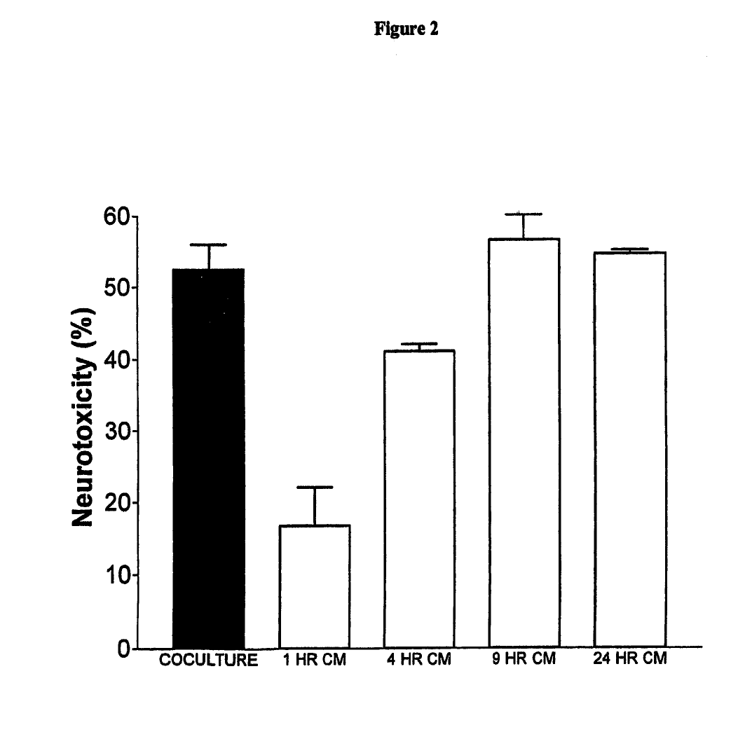

The accumulation of a neurotoxic factor, now referred to as EDTF^{MV}, in conditioned medium plateaued after about 9 h of exposure to microvessels and remained constant for at least 24 h (FIG. 2). Microvessel-conditioned media, collected between 4 and 9 h, evoked neuronal cell death comparable to that of AD microvessels in co-culture for 4 h, demonstrating that the neurotoxicity of AD microvessels was due to release of a soluble factor and that similar neuronal responses were obtained with either the conditioned medium or co-culture assays (FIG. 2). These results demonstrate that the lethal effect of AD microvessels is not dependent on a feed back loop between neurons and microvessels, since microvessel-conditioned medium is also neurotoxic.

Microvessel-derived neurotoxicity is dose dependent.

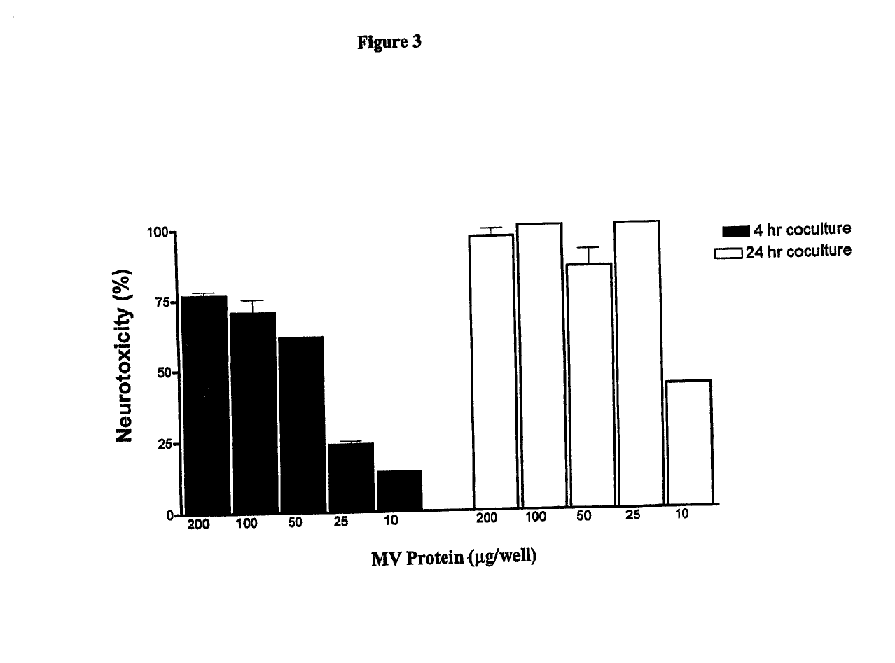

Our initial quantitation of vascular-derived neurotoxicity is shown in FIG. 3. Addition of increasing concentrations of the MV preparation (10 μg-200 μg protein) showed a dose-dependent increase in LDH release from neuronal cultures when measured at 4 hours. At 24 hours there was a massive cytotoxic response evoked in the neuronal cultures with concentrations of MV protein as low as 25 μg. Cultures of either MVs or neurons alone showed relatively little LDH release (data not shown).

AD microvessel cytotoxicity is neurospecific.

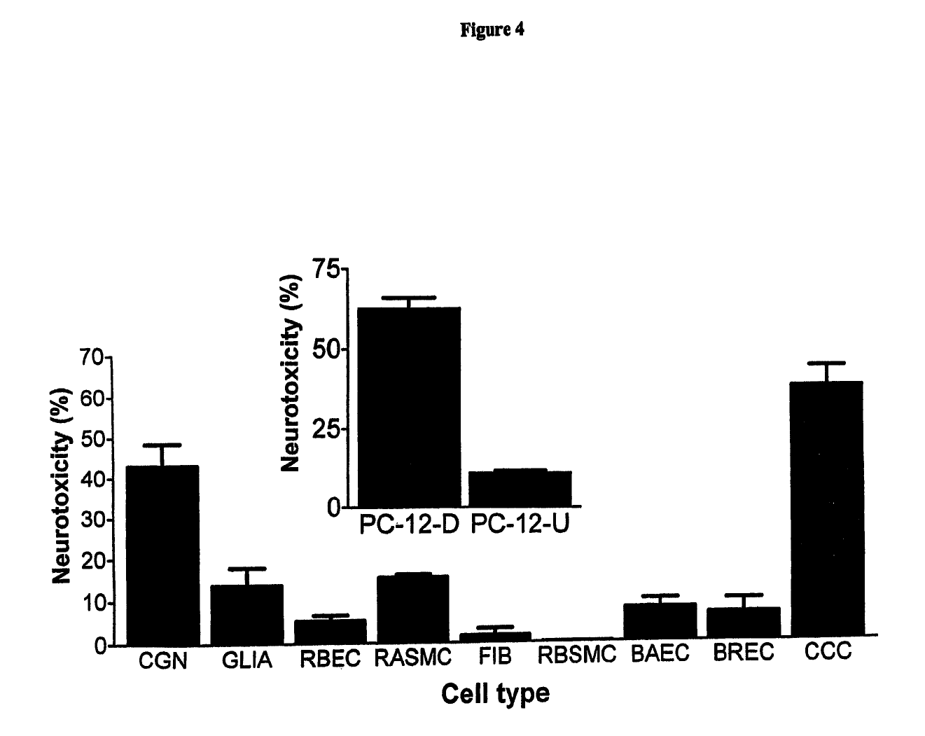

Although exposure to AD microvessels kills primary neuronal cultures and primary cerebellar granule neurons, the viability of 7 non-neuronal cell types, including brain-derived glia, was unaffected (FIG. 4). Other evidence that AD microvessel EDTF is neurospecific comes from experiments showing that the differentiated PC-12 neuronal cell line is killed in the presence of AD microvessels, but there is no cytotoxic effect on the same cell line when PC-12 cells are undifferentiated and therefore non-neuronal (FIG. 4, inset).

The vascular-derived neurotoxic factor is trypsin-sensitive and heat-labile.

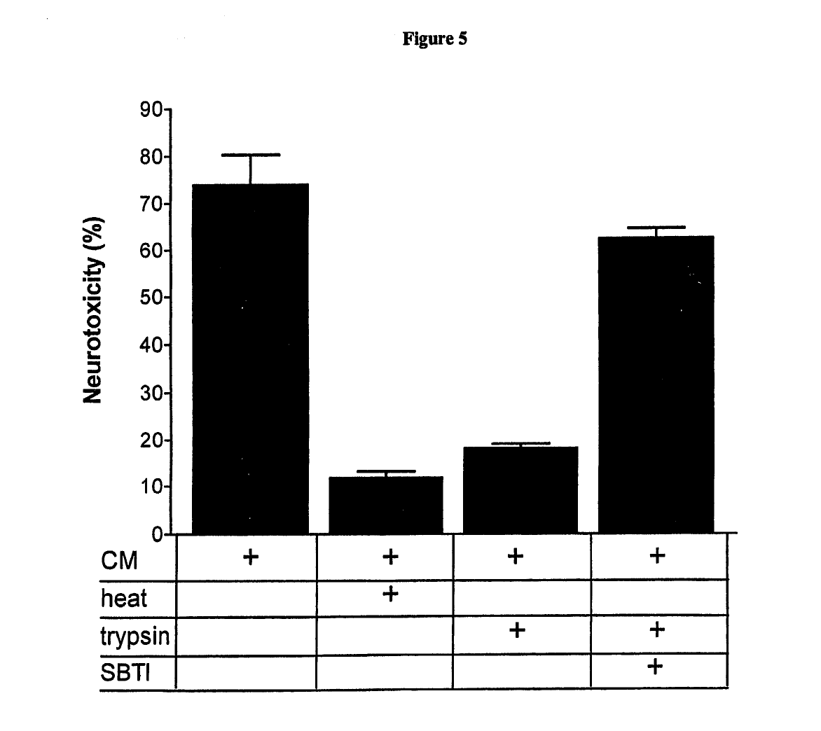

Initial experiments ruled out the involvement of several possible candidate molecules as EDTF^{MV}. Nitric oxide was not responsible for the microvessel-mediated neurotoxicity, because preincubation of microvessels with the nitric oxide synthase inhibitor 10 μM N^{G}Nitro-L-arginine, a concentration previously shown to inhibit microvascular nitric oxide production, did not affect toxicity of the conditioned media (% neurotoxicity of conditioned medium=65±7; media with N^{G}Nitro-L-arginine=69±12). Furthermore, EDTF^{MV} is a protein because the neurotoxicity activity is both trypsin-sensitive and heat labile (FIG. 5). Further, incubation of Alzheimer's disease microvessels with cycloheximide (10 μg/ml) inhibited appearance of the neurotoxic factor as assessed by a 60% decrease in neuronal necrosis. EDTF was shown not to be amyloid β or tumor necrosis factor α because they induced little neuronal necrosis at 4 h and neutralizing antibodies to these proteins did not significantly affect EDTF^{MV} neurotoxicity (data not shown).

Neuronal cell death occurs by either apoptosis or necrosis and is microvessel dose- and time-dependent.

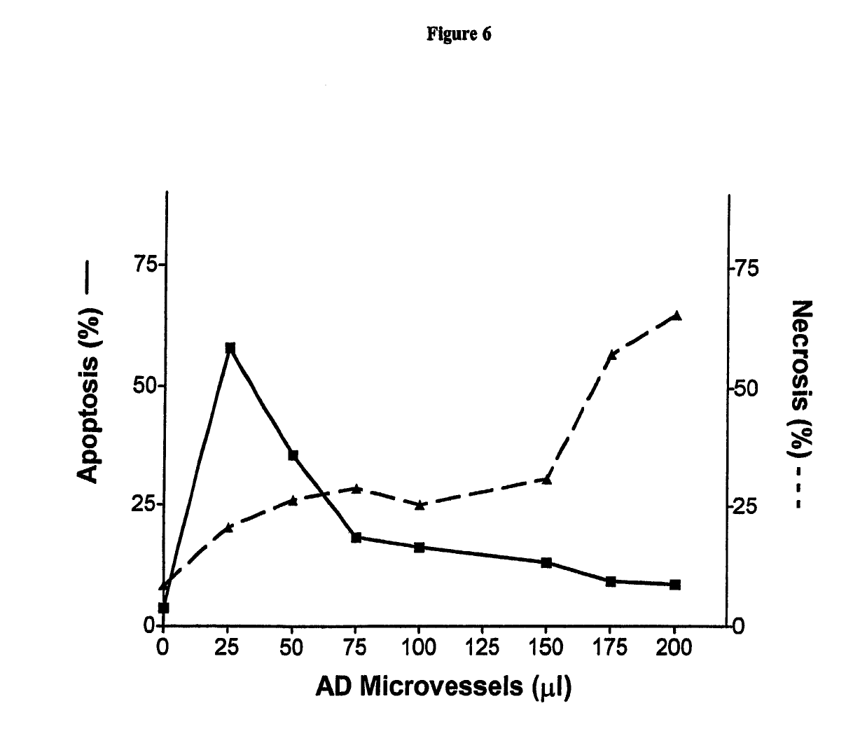

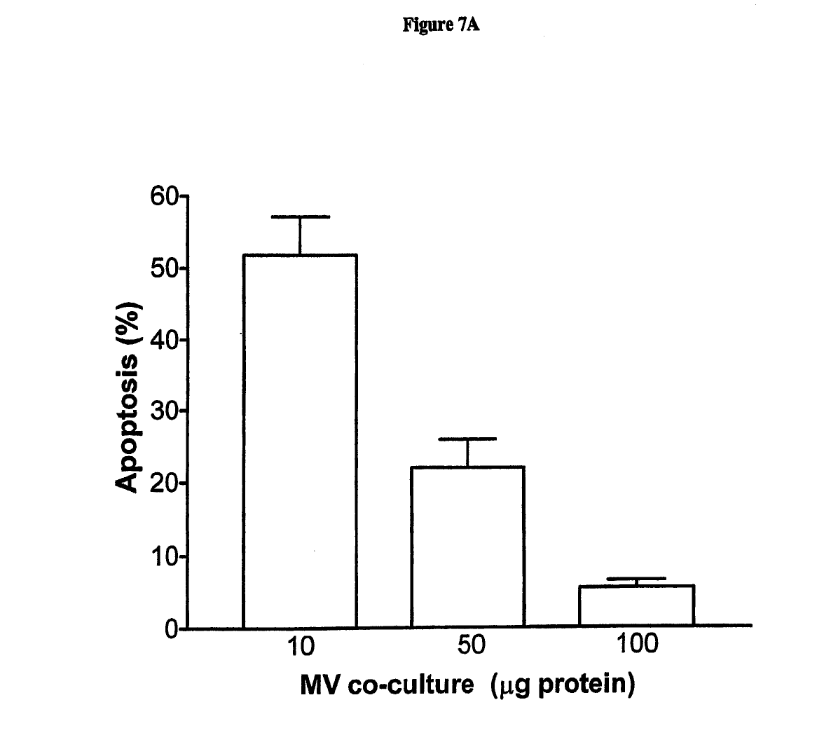

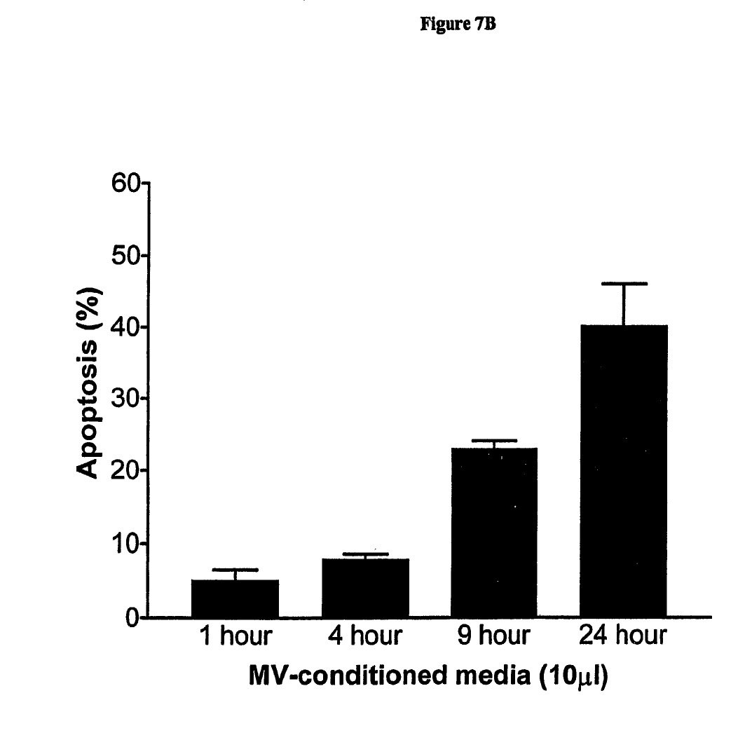

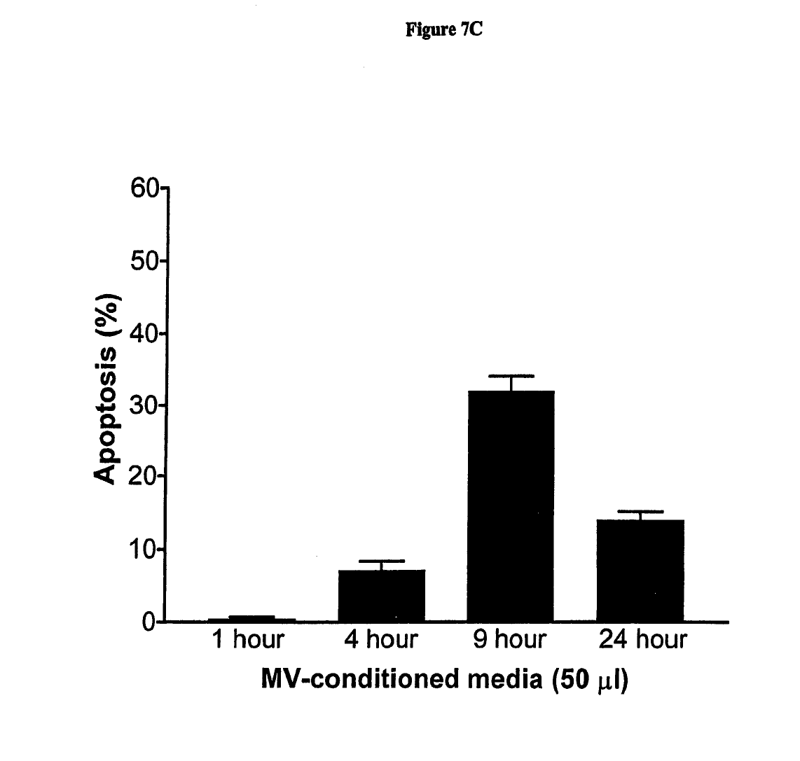

The very short time required for cell killing (<4 h), as assessed by the release of LDH, was consistent with necrosis. Since neurons can undergo either necrosis or apoptosis, depending on the intensity of the insult (Bonfoco et al., “Apoptosis and Necrosis: Two Distinct Events Induced, Respectively, By Mild And Intense Insults With N-methyl-D-aspartate Or Nitric Oxide/Superoxide In Cortical Cell Culture”, Proc Natl Acad Sci USA, 92:7162, 1995), the mechanism of cell death evoked directly by AD microvessels or by microvessel-conditioned media was examined over a wide concentration range. The data show that AD microvessels can evoke either neuronal apoptosis or necrosis and the balance between these two pathways is reciprocal and dose-dependent. High concentrations of conditioned media from AD microvessels caused predominately necrosis, whereas apoptosis was more prominent at lower concentrations (FIG. 6). Co-culture with 10 μg AD vessel protein caused a 9-fold higher level of neuronal apoptosis than co-culture with 100 μg (FIG. 7A). Ten μl of medium conditioned by exposure to AD microvessels required 24 h to evoke a maximal apoptotic response in neurons (FIG. 7B), while the time necessary for 50 μl of conditioned medium from AD vessels to elicit maximal neuronal apoptosis was only 9 hrs (FIG. 7C). The apoptotic response was less after exposure to 50 μl (FIG. 7C) compared to 10 μl (FIG. 7B) of AD-conditioned medium because of the greater necrotic response at the higher dose of conditioned medium. These data suggested that neurotoxic activity was dependent on both the amount of microvessel protein and the duration of medium conditioning.

### Results Using Cultured Rat Brain Endothelial Cells

Rat brain endothelial cells were studied to examine their effects on neuronal cell viability. As shown herein, inhibition of endothelial Protein Kinase C (PKC) results in the production of a factor EDTF^{BIM} that is toxic to neurons in culture. Application of EDTF^{BIM} causes lethal injury, determined by release of LDH, to neuronal cell cultures, in a dose-dependent manner, within 2 hours. This protein mediator is stable after concentration (>5-fold) and addition of glycerol (to 10%). EDTF^{BIM} can be stored frozen (−70° C.) or at 4° C. for at least 8-10 days.

Cell cultures:

Small arterioles from rat brain were isolated, and endothelial cell cultures initiated, as previously published (Diglio C. A., Grammas P., Giacomelli F. and Wiener J., “Rat Cerebral Microvascular Smooth Muscle Cells in Culture”, J. Cell Physiol., 129:131-141, 1986; Diglio C. A., Liu W., Grammas P., Giacomelli F., and Wiener J., “Isolation and Characterization of Cerebral Resistance Vessel Endothelium in Culture”, Tissue and Cell, 25:833-846, 1993). We have previously demonstrated the endothelial nature of these cells directly, by angiotensin converting enzyme reactivity and the uptake of labeled low density lipoproteins, as well as indirectly, by lack of reactivity to antibodies for smooth muscle myosin and α-actin. Cells (passages 10-15) were maintained in Dulbecco's modified Eagle's medium (DMEM) and 10% fetal calf serum and subcultured using trypsin-versene (0.025%).

For the preparation of endothelial-conditioned media, the media were removed and confluent endothelial cell cultures washed with versene and fresh serum-free DMEM containing (unless otherwise indicated) 1 μM of the PKC inhibitor, bisindolylmaleimide (BIM), was added. The conditioned medium was collected, after 8-24 hrs, and centrifuged at 100,000 g to remove debris and suspended cells, and 5% heat-inactivated horse serum was added prior to application of media to neuronal cultures.

Neurons were obtained and cultured from 15-18 day rat embryos by published methods (Dawson V. L., Dawson, T. M., London E. D., Bredt D. S., Snyder S. H., “Nitric Oxide Mediates Glutamate Neurotoxicity in Primary Cortical Cultures”, Proc. Natl. Acad. Sci. USA, 88:6368-6371, 1991, with our modifications: Grammas P, Moore P, Weigel P H, “Production of Neurotoxic Factors by Brain Endothelium in AD”, Ann. NY Acad. Sci., 826, 1997). Cerebral cortices were isolated, dissociated in Brooks-Logan solution (5% phosphate buffered saline, 0.04 M sucrose, 10 mM HEPES, pH 7.5, 0.03 M glucose) by trituration and seeded in polylysine-coated 24 well plates (15), containing DMEM with 5% heat-inactivated horse serum. After five days of culture, the cells were treated with 10 μg of 5-fluoro-2′-deoxyuridine per well and cultures were maintained for an additional 2 to 3 weeks. Immunohistochemistry using an antibody against neuron specific enolase and fluorescence activated cell sorting of these cultures indicated that more than 80% were neuronal.

Determination of neuronal cell death by necrosis and apoptosis was as described on pages 17 and 18.

### Results

The application of conditioned media from cerebrovascular endothelial cell cultures (containing EDTF^{BIM}), collected 24 hrs after treatment with the PKC inhibitor BIM (1 μM), caused lethal injury to over 90% of the neurons in culture (Table 1).

TABLE I
Appearance of EDTF^{BIM} in response to endothelial PKC inhibition

| Cells | BIM | % Cytotoxicity |
|---|---|---|
| Neurons | + | 91.80 ± 8.20 |
| Neurons | − | 13.97 ± 0.83 |

Twenty-four hour conditioned media were collected from endothelial cell cultures treated with bisindolylmaleimide (+) or media alone (−), centrifuged at 100,000 g and 5% heat inactivated horse serum added. The samples were then applied to mature neuronal cultures, the media removed after 24 hrs, and assayed for LDH. The cytotoxicity (LDH release) is expressed as % of total LDH released after treatment with Triton X-100.

Media collected from confluent, untreated endothelial cultures did not affect neuronal viability (14% cytotoxicity) (Table 1). In addition, BIM had no toxic effect on endothelial cultures (10% cytotoxicity) or when added directly to neuronal cultures (16% cytotoxicity). The EDTF^{BIM} did not cause lethal injury when added to other endothelial cells (such as aorta-derived), smooth muscle cells, fibroblasts or glial cells. In addition, non-endothelial cell-types, including fibroblasts and smooth muscle cells, did not produce a toxic mediator after PKC inhibition (data not shown).

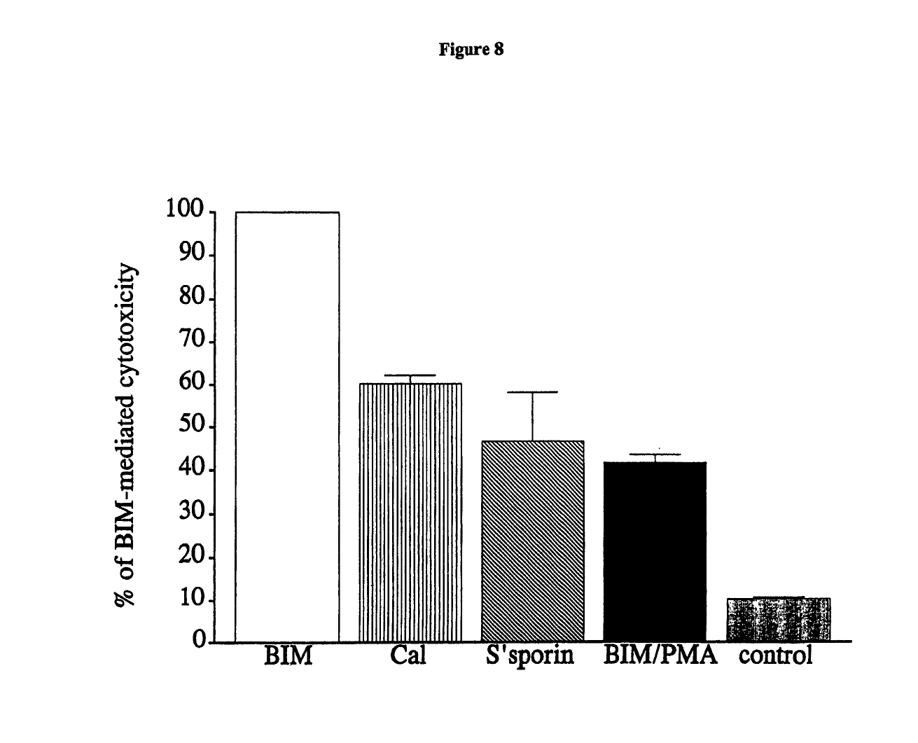

To confirm that production of EDTF^{BIM} by endothelial cells in response to BIM was indeed related to PKC inhibition, the effects of the PKC agonist, phorbol myristate acetate (PMA) as well as other PKC inhibitors, calphostin and staurosporine, were explored. Concurrent incubation of PMA and BIM reduced the amount of neuronal cytotoxicity (i.e. EDTF^{BIM} release) approximately 60% compared to the response evoked by BIM alone (FIG. 8). Treatment of endothelial cell cultures with other PKC inhibitors, calphostin and sphingosine, also elicited EDTF^{BIM} release. Calphostin and staurosporine, less potent inhibitors of PKC than BIM, evoked 60 and 47%, respectively of the cytotoxic response elicited by BIM (FIG. 8).

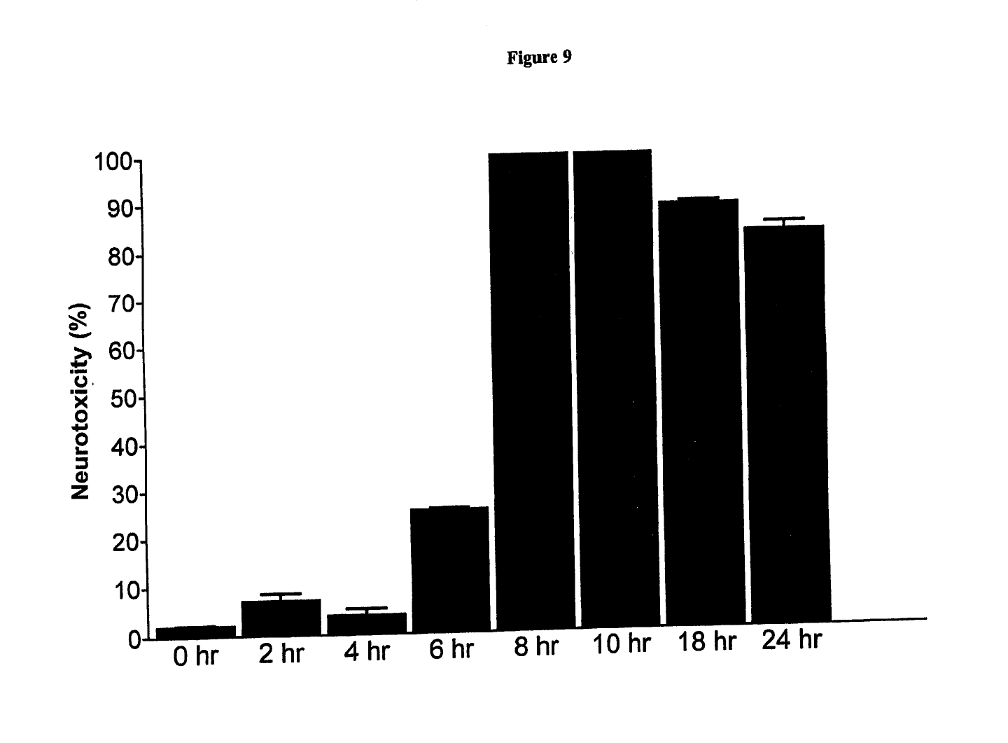

To determine the kinetics of EDTF^{BIM} appearance, endothelial cells were treated with BIM and media samples were collected at timed intervals. FIG. 9 shows that the neurotoxic mediator was not present before 8 hours. EDTF^{BIM} production by endothelial cells may therefore require gene expression.

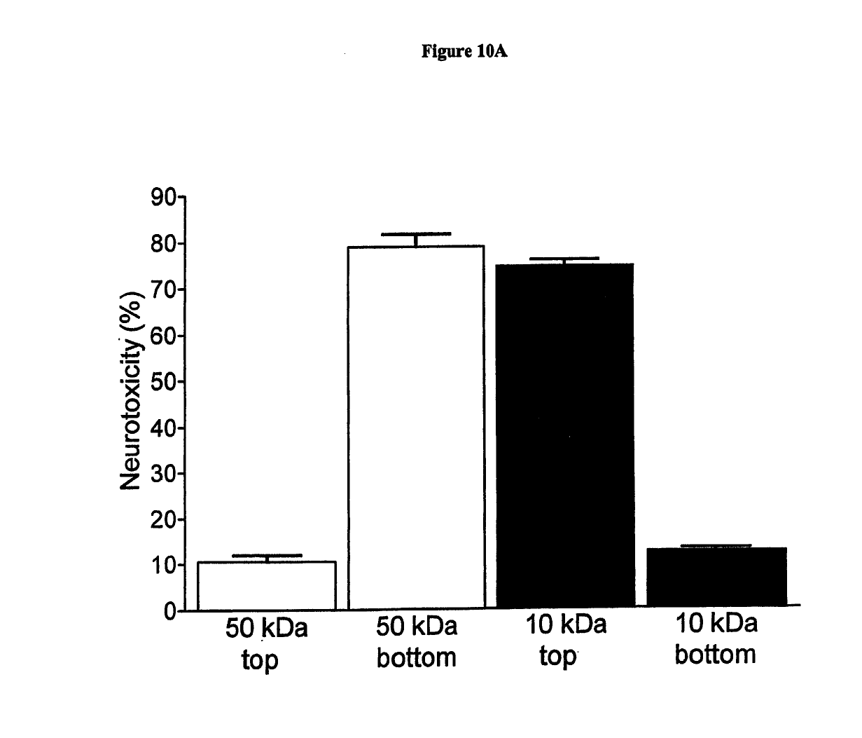

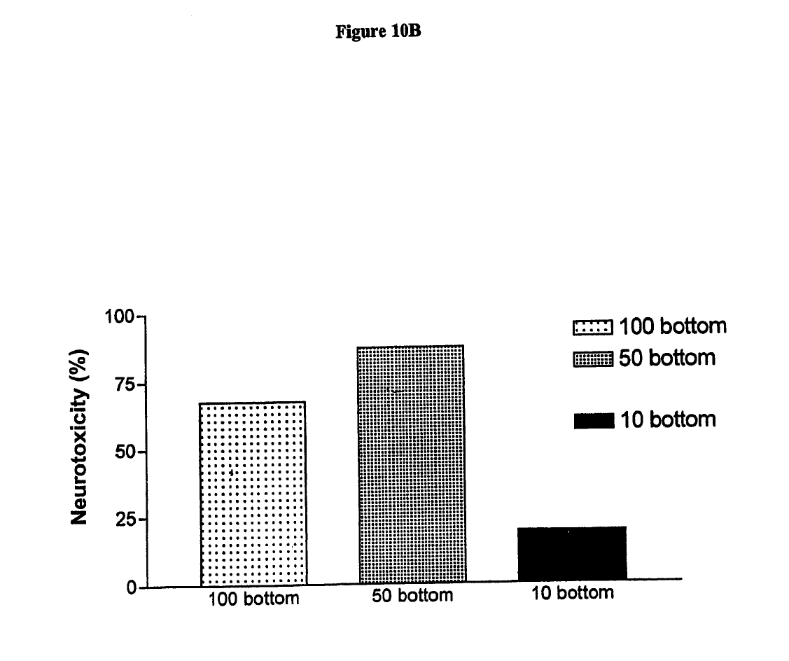

The EDTF^{BIM} protein is soluble (non-sedimenting at 100,000 g for 1 hr), heat-labile, susceptible to proteolysis (i.e. using trypsin and chymotrypsin) and loses activity with repeated freeze-thawing (data not shown). Fractionation of media containing EDTF^{BIM}, using CENTRICON™ devices (Amicon, Beverly, Mass.) with 10 or 50 kDa MW cut-offs, indicated that most of the activity was located in the bottom fraction of the 50 kDa device , i.e. EDTF^{BIM} is < 50,000 MW and in the top fraction of the 10 kDa CENTRICON™ device; therefore EDTF^{BIM} m is >10,000 MW (FIG. 10A). EDTF^{MV} is >10,000 MW and <50,000 MW also (FIG. 10B). Further isolation of the media containing EDTF^{BIM} or EDTF^{MV} leading to purified EDTF^{BIM} or EDTF^{MV} is well within the ability of a person of ordinary skill in the art, as described elsewhere herein.

### Monoclonal Antibodies

Preparation of endothelial-conditioned medium containing EDTF(s).

Endothelial cultures were maintained at confluence in DMEM containing 10% FCS for 2 days. The culture medium was then removed, the cells washed with versene buffer (three 5-10 minute washes) to remove residual serum, and fresh serum-free DMEM containing 1 μM BIM and 0.1% lactalbumin hydrolysate was added. The conditioned medium was then collected after 8-24 hours and centrifuged at 500×g to remove debris and suspended cells. Conditioned media was passed through a 50 kDa CENTRICON™ and the flow-through was then concentrated using a 10 kDa CENTRICON™. Samples were sterile filtered with a 0.4 μm syringe filter and used for immunization.

Monoclonal antibody production:

Balb/c mice (Jackson Labs, Bar Harbor, Me.) were immunized 3 times (intraperitoneal) each with CENTRICON™ concentrated-EDTF^{BIM} with Freund's complete adjuvant (100 μl per injection) at two week intervals. After a two-month rest, they were reinjected with concentrated EDTF^{BIM} and the fusion of spleen lymphocytes was performed 3 days later. A myeloma cell line (1.5×10⁷ viable cells ) was mixed with 1.7×10⁸ viable spleen cells from one animal and fused by standard procedures using PEG 1500. Approximately 12 96-well plates were seeded with cells from each fusion. After selection in hypoxanthine/aminopterin/thymidine selective media, the supernatants from the surviving hybridoma wells were screened in an ELISA assay to identify wells producing antibody to the EDTF-containing concentrate (antigen). Wells that were consistently positive in the ELISA screen (using the initial antigen) were then assayed for their ability to immunoprecipitate EDTF and therefore inhibit the neurotoxic activity of the EDTF in our neuronal bioassay system. Positive hybridoma wells were subcloned by limiting dilution to isolate multiple individual clones. Five hybridoma clones that significantly inhibited EDTF activity by immunoprecipitation of the EDTF protein were used for preparation of ascites. For production of ascites fluid, approximately 5×10⁶ hybridoma cells were injected per Balb/c mouse 2 weeks after Pristane treatment. Monoclonal antibodies were isolated from ascites fluid by protein G affinity chromatography, eluted and immobilized by binding to protein A Sepharose and used for the immunoprecipitation of EDTF.

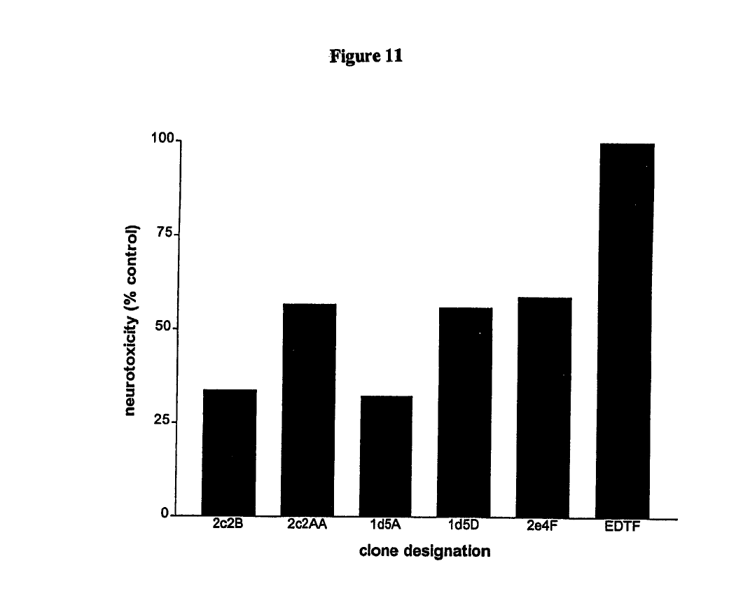

The five hybridoma cultures that secrete monoclonal antibodies that can immunoprecipitate, and therefore reduce, EDTF toxicity in our bioassay are identified as 2c2B, 2c2AA, 1d5A, 1d5D and 2e4F. As indicated in FIG. 11, antibody from these five clones reduce EDTF toxicity (expressed as 100%) by 41 to 67%.

Monoclonal antibody from hybridoma line 2e4f was isolated from ascites fluid by protein G affinity chromatography, eluted and immobilized again by binding to protein A Sepharose. The antibody-protein A sepharose conjugates were then added to supernatants from cultures of Alzheimer's disease patients' brain microvessels (i.e., supernates containing EDTF^{MV}) (MV) or rat brain endothelial cells that had been cultured for 24 hours in serum free medium alone containing the protein kinase C inhibitor bis-indolylmaleimide (i.e., medium containing EDTF^{BIM}) (CM⁺). After overnight incubation, the antibody-protein A sepharose conjugates were removed by centrifugation and the treated culture supernatants were added to cultured rat brain neurons to assay for neurotoxic activity. After 24 hours, lactate dehydrogenase (LDH) activity in the medium and total lactate dehydrogenase activity, released after lysing the cells with detergent, were determined. Neurotoxicity is expressed as percent lactate dehydrogenase activity released prior to cell lysis. The background value for the cm− samples (≈15%) was then subtracted. The present invention preferably comprises or contemplates the use of antibodies (monoclonal or specific polyclonal) which bind to EDTF or fragments thereof having a K_{D}< 10⁻⁸M.

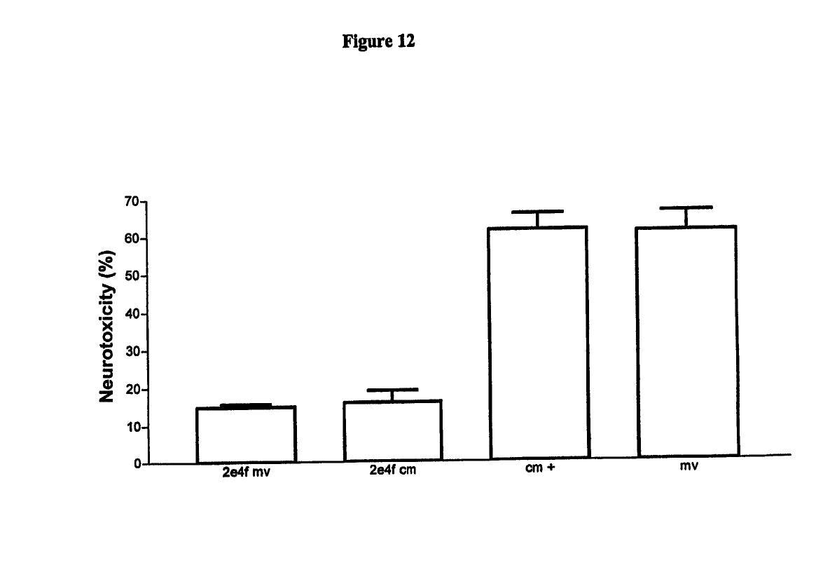

FIG. 12 indicates that the neurotoxic activities of EDTF^{BIM} contained in rat endothelial cell conditioned medium containing BIM (CM⁺) and of EDTF^{MV} contained in AD brain microvessel culture medium (MV) are virtually identical. Further, the figure indicates that monoclonal antibody from hybridoma line 2e4F has a virtually identical effect on removing the activity of EDTF^{BIM} and EDTF^{MV} providing further evidence that the EDTF^{BIM} and EDTF^{MV} proteins are substantially similar and act by similar mechanisms.

### Isolation and Purification of EDTF

As noted, monoclonal antibodies that recognize, bind to and immunoprecipitate endothelial derived toxic factor, or EDTF, have been identified. These antibodies enable the isolation of EDTF by “affinity” immunopurification as follows. Medium conditioned by isolated brain microvessels from AD patients or cell culture systems in vitro, or any other fluid containing EDTF, can be used as a starting material. For example, the conditioned media is collected from endothelial cell cultures treated in vitro with an appropriate inhibitor that induces the production of EDTF, such as the Protein kinase C inhibitor BIM as discussed elsewhere herein. A variety of different blood vessel endothelial cell cultures are suitable for the generation of EDTF, including those isolated and established in tissue culture from the small or large blood vessels of most mammalian species such as humans and rats. Routinely, one might use human endothelial cell cultures established from heart, lung or brain blood vessels.

Because the molecular size of the EDTF is between 10-50 kDa, it is preferable to enrich this activity prior to the final purification. For example, the EDTF activity in the conditioned media of endothelial cells exposed to BIM can be fractionated by centrifugation through CENTRICON™ devices (from Amicon Inc.) with a 50,000 molecular weight cut-off and the flow-through material then concentrated by centrifugation over a 10,000 molecular weight cutoff CENTRICON™ device. The resulting concentrate, which is typically enriched in EDTF activity about 10-fold, is then passed over a column containing one or more of the monoclonal antibodies against EDTF described herein that have been covalently attached to the chromatographic support (e.g. beads of an agarose-based resin). Other antibodies which are effective against EDTF but are not described herein may also be used.

A variety of protein immobilization procedures would provide useful affinity adsorbents for this purpose, including the use of CNBr-activated Sepharose or Affi-Gel (Pierce Chemical Co.) In the latter embodiment, the antibodies are covalently attached in an oriented manner by means of their oligosaccharide chains so that the antigen combining regions are unhindered and free to react with the EDTF molecules. Proteins not bound to the monoclonal antibody column will be removed in the flow-through and by extensive washing of the column. EDTF molecules that are specifically bound to the monoclonal antibody affinity column are then subsequently released and eluted, usually by one or more of the following agents: low pH (typically 0.1 M glycine buffer, pH 2.3), high pH (typically 0.1 M sodium carbonate or triethylamine buffer, pH 11.5), high magnesium salt (4M MgCl₂), organic solvents (such as 10% dioxane or 50% ethylene glycol) or chaotropic agents (such as 3M sodium thiocyanate).

Purification of the EDTF to homogeneity by immuno-affinity chromatography is typically verified by analysis of samples using sodium dodecyl sulfate-polyacrylamide gel electrophoresis, (SDS-PAGE). Further confirmation that the EDTF has been obtained is achieved by Western blot analysis. After SDS-PAGE and electroblotting to transfer proteins to nitrocellulose or polyvinylidene difluoride membranes, the protein bands are visualized by reaction with a primary antibody against the EDTF followed by a secondary antibody conjugate. Typically, a different specific monoclonal antibody against the EDTF will be used than that used for the immuno-purification. The secondary antibody could be a variety of second-species antibodies (e.g. rabbit or goat), against the appropriate idiotype of the mouse monoclonal antibody, conjugated to a recorder molecule, such as the enzymes alkaline phosphatase or horseradish peroxidase capable of developing a stain to visualize the presence of EDTF.

The primary amino acid sequence of EDTF can be obtained by standard procedures after SDS-PAGE of the pure protein. The EDTF protein bands can be stained, excised, and the gel samples sent to a Protein Chemistry Laboratory for in-gel tryptic digestion and amino acid sequencing of individual peptides after purification by reverse phase HPLC. Standard molecular biology and polymerase chain reaction techniques for cloning the cDNA encoding EDTF can be employed to obtain the nucleic acid sequence encoding the EDTF protein.

In the present study, we have shown that inhibition of endothelial cell PKC results in the production of a molecule (EDTF^{BIM}) with toxic properties for neuronal cells in culture. Initial experiments on the nature of this molecule indicate the following: (i) it is a soluble protein with a MW between 10-50 kDa; (ii) it requires approximately 8 hours to appear in the medium after treatment of endothelial cells; (iii) it causes lethal injury to neuronal cells by 2 hrs; (iv) it is specific for neurons; (v) this mediator is not TNFα, and (vi) the EDTF^{MV} derived from human brain microvessels and EDTF^{BIM} derived from rat brains have essentially identical neurotoxic functions and are substantially similar proteins.

The cytotoxic response to EDTF^{BIM} is variable in magnitude (between 60 and 90%). We believe this variability may be explained, in part, by differences in endothelial cell number. While all EDTF^{BIM} collections are made from confluent endothelial cell cultures, even at confluence the endothelial cell density in these cultures varies between 3.5 to 6.9×10⁶ in 100 mm plates. Indeed, other work confirms that the production of EDTF is closely linked to endothelial cell number. Alternatively, since the cytotoxicity of EDTF^{BIM} is evaluated on primary cerebral cortical cultures that are predominately (>80%), but not exclusively, neuronal, and EDTF^{BIM} is not toxic to glial cells or other non-neuronal cells, this may also account for the variability of the cytotoxicity of EDTF^{BIM}.

Attempts to understand the pathogenesis of AD have focused on the development of senile plaques and neurofibrillary tangles and how these lesions contribute to neuronal cell loss. Here we describe for the first time direct neuronal cell death mediated by isolated blood vessels from AD patients.

Since neuronal cell loss underlies the dementia of AD, identification of factors that cause lethal neuronal injury is central to understanding the pathogenesis of this disease and ultimately to developing effective therapies. Our present finding that co-culture of AD microvessels with neurons or addition of microvessel-conditioned medium to neurons causes neuronal cell death identifies the cerebral vasculature as a novel source of neurotoxic factors in the brains of AD patients. When endothelial cells are activated or injured they produce both superoxide and hydroxyl radicals as well as nitric oxide (Nakazano et al, “Does Superoxide Underlie The Pathogenesis of Hypertension?”, Proc Natl Acad Sci, 88:10045-10048, 1991; Kumar et al., “Anoxic Injury Of Endothelial Cells Increases Production Of Nitric Oxide And Hydroxyl Radicals”, Biochem Biophys Res Comm, 219:497-501, 1996). However, the vascular-derived factor claimed herein only kills primary cortical neurons, primary cerebellar granule neurons and the differentiated (i.e., neuronal) PC-12 cell line. This factor released by microvessels does not kill non-neuronal cell types. EDTF is therefore not general, but rather very neurospecific.

Necrosis and apoptosis are two distinct mechanisms of cell death differing in their effects on cellular morphology and metabolism. Necrosis is usually evoked by intense insults and is characterized by cell swelling, membrane lysis, injury to cytoplasmic organelles, and release of cellular contents. The apoptotic cell death program is defined by cell shrinkage, membrane blebbing, nuclear pyknosis, chromatin condensation and genomic fragmentation (Manjo et al., “Apoptosis, Oncosis And Necrosis: An Overview Of Cell Death”, Am J Pathol, 146:3-15, 1995). While necrosis and apoptosis are distinct processes, they may also represent extremes of a cell death continuum that is dependent on the intensity and duration of the stimulus as well as the status of the target cell. Our results show that the EDTF evokes either necrosis or apoptosis depending on the microvessel dose. This finding is consistent with the data from experiments of others (Bonfoco et al., “Apoptosis and Necrosis: Two Distinct Events Induced, Respectively, By Mild And Intense Insults With N-methyl-D-aspartate Or Nitric Oxide/Superoxide In Cortical Cell Culture”, Proc Natl Acad Sci USA, 92:7162, 1995) using other neurotoxic agents, such as N-methyl-D-aspartate and nitric oxide, that also evoke both apoptotic and necrotic patterns of cell death (Bonfoco et al., 1995).

EDTF is a protein, which makes it very likely that the mechanism of action involves a receptor-mediated neuronal response. These results demonstrate that the vascular-derived neurotoxic factor EDTF is an important new paradigm of neuronal injury in AD.

### Utility

### Methods of Diagnosis and Detecting EDTF in a Fluid

The monoclonal antibodies described herein (or others effective against EDTF but not described herein for example, antibody which demonstrates an immunological binding characteristic of monoclonal antibody produced by at least one of hybridomas 2c2B, 2c2AA, 1d5A, 1d5D, and 2e4F and/or which preferably have a K_{D}<10⁻⁸M) which are able to recognize and bind to EDTF^{BIM} or EDTF^{MV}, can be used in a variety of assays to detect the presence of the EDTF protein or its breakdown products in bodily fluids such as serum, cerebral spinal fluid or urine thereby enabling antemortem detection of Alzheimer's disease. Reaction of an EDTF-recognizing antibody with EDTF in serum or cerebrospinal fluid can be demonstrated in Western blot analyses as noted above or in a dip stick format in which case the antibody to EDTF would be coupled to the paper support and color development would be used to visualize the presence or absence of EDTF in the body fluid. In addition, these antibodies could be used on brain sections at autopsy to confirm the diagnosis of Alzheimer's disease. Detection methods using such monoclonal antibodies in such a manner are well known to those of ordinary skill in the art.

As noted, the present invention includes methods of detecting EDTF or fragments thereof in vivo in a sample of the serum or cerebrospinal fluid of a subject. For example, antibodies specific for animal or human EDTF or fragments thereof, may be detectably labeled with any appropriate ligand, for example, a radioisotope, an enzyme, a fluorescent label, a paramagnetic label, or a free radical. Methods of making and detecting such detectably labeled antibodies or their functional derivatives are well known to those of ordinary skill in the art.

The detection of foci of such labeled antibodies may be indicative of neurological sites affected by Alzheimer's disease. In a preferred embodiment, this technique is accomplished in a non-invasive manner through the use of magnetic imaging, or fluorography, for example. For example, such a diagnostic test may be employed to determine a subject's clinical status in Alzheimer's disease.

One of the ways in which the EDTF-specific antibody can be detectably labeled is by linking the same to an enzyme. This enzyme, in turn when later exposed to its substrate, will react with the substrate in such a manner as to produce a chemical moiety which can be detected, for example, by spectrophotometric, fluorometric or by visual means. Enzymes which can be used to detectably label the EDTF-specific antibody include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholine esterase.

The EDTF-specific-antibody may also be labeled with a radioactive isotope which can be determined by such means as the use of a gamma counter or a scintillation counter or by audioradiography. Isotopes which are particularly useful for the purpose of the present invention are: ³H, ¹²⁵I, ¹³¹I, ³⁵S, ¹⁴C, and ⁵¹Cr.

It is also possible to label the EDTF-specific antibody with a fluorescent compound. When the fluorescently labeled antibody is exposed to light of the proper wave length, its presence can then be detected due to the fluorescence of the dye. Among the most commonly used fluorescent labelling compounds are fluorescein isothiocyanate rhodamine, Texas Red, phycoerytherin, phycocyanin, allophycocyanin, o-phthaldehyde and fluorescamine.

The EDTF-specific antibody can also be detectably labeled using fluorescence emitting metals such as ¹⁵²Eu, or others of the lanthanide series. These metals can be attached to the EDTF-specific antibody using such metal chelating groups as diethylenetriaminepentaacetic acid (DTPA) or ethylenediaminetetraacetic acid (EDTA).

The EDTF-specific antibody also can be detectably labeled by coupling it to a chemiluminescent compound. The presence of the chemiluminescent-tagged EDTF-specific antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the EDTF-specific antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Detection of the EDTF-specific antibody may be accomplished by a scintillation counter, for example, if the detectable label is a radioactive gamma emitter, or by a fluorometer, for example, if the label is a fluorescent material. In the case of an enzyme label, the detection can be accomplished by calorimetric methods which employ a substrate for the enzyme. Detection may also be accomplished by visual comparison of the extent to enzymatic reaction of a substrate in comparison with similarly prepared standards.

All of the assay methods listed herein are well within the ability of one of ordinary skill in the art given the teachings provided herein.

### Methods of Screening for Agents Which Inhibit EDTF Activity or Synthesis

To screen for any compounds of interest that inhibit the synthesis and/or effects of EDTF, one can employ established cell cultures of endothelial cells, such as the EC150 cells and established cell cultures of human neuronal cells such as the HCN-1 cells. Agents that block the killing of neurons by EDTF can be identified by assessing their protective effect on HCN-1 cells exposed to a known amount of EDTF, for example, sufficient to cause approximately 50% cell death, either by apoptosis or necrosis, within 48 hours after exposure. Such agents would be incubated with the neuronal cultures alone at comparable concentrations to ensure that they are not toxic to the neurons. Agents that protect the neurons from cell death will be evident in such assays when the expected level of 50% cell death is significantly decreased.

Preferably, one would try to select agents able to penetrate the blood brain barrier, in particular, lipophilic molecules or compounds conjugated to proteins or other small molecules whose transport across the endothelium is mediated by membrane transporters, so-called carriers.

Effects on the cytotoxicity of EDTF can be measured by release of the enzyme lactate dehydrogenase, since this a relatively easy enzyme to assay and one which is generally regarded to reflect cell disruption and death when it is released into the medium. In vivo, in patients it is anticipated that most of the toxic effects of EDTF on neurons will be mediated by a slower apoptotic process rather than direct necrosis. The findings in the present application support the idea that EDTF is able to exert toxic effects on neurons by either necrotic mechanisms and/or apoptotic mechanisms (or programmed cell death), depending on the concentration of EDTF to which the neurons are exposed. At high concentrations of the EDTF, neurons are killed very quickly, within a few hours, by necrotic processes, whereas at increasingly lower concentrations, the neurons are triggered to undergo the cell death program of apoptosis. Compared to necrosis, the apoptotic process usually takes many more hours to days, before most cells in a culture are dead. Screening for agents that delay or prevent apoptosis, would employ standard assays known to those in the field, such as the TUNEL staining assay, Annexin V staining (to detect the presence of phosphatidyl serine on the surface of cells), and/or ELISA-based assays for the formation of nucleosomes.

In one embodiment, the screening method comprises providing a sample of neuronal cells, treating the cells with a test compound, contacting the treated cells with EDTF, and examining the cells for evidence of apoptosis or necrosis and wherein when apoptosis or necrosis of the cells fails to be observed or is reduced, concluding that the test compound inhibits apoptosis or necrosis of neuronal cells by EDTF.

Although EDTF^{BIM} is derived from the rat, EDTF^{BIM} is able to kill human neurons in culture. Human HCN-1 neuronal cells can, therefore be employed with EDTF^{BIM} to screen for agents that are able to protect the human neurons from apoptosis induced by EDTF^{MV}.

All of the assay methods listed herein are well within the ability of one of ordinary skill in the art given the teachings provided herein.

### Therapy

The present invention contemplates a method for the treatment of subjects afflicted with Alzheimer's disease by the administration of an effective amount of a therapeutic agent which inhibits EDTF activity or synthesis in vivo.

After derivation of the primary amino acid sequence of EDTF or of the nucleic acid sequence encoding EDTF, blocking peptides which bind to the EDTF receptor or antisense oligonucleotides based on the EDTF coding region can be prepared. Such peptides, or antisense oligonucleotides can be delivered via a variety of possible routes including intravenous injection at distant sites or directly into the carotid artery, in a subepidermal time released form, or via implanted drug release pumps. Such peptides, or antisense oligonucleotides delivered to and taken up by brain endothelial cells, will inhibit binding of EDTF to neurons, or vascular synthesis of EDTF respectively, thereby alleviating symptoms of AD and/or slowing the progression and development of the AD syndrome. Other therapeutics contemplated herein are antibodies such as antibody from hybridomas 2c2B, 2c2AA, 1d5A, 1d5D, and 2e4F and antibody which demonstrates an immunological binding characteristic of monoclonal antibody produced by at least one of hybridomas 2c2B, 2c2AA, 1d5A, 1d5D, and 2e4F, or fragments thereof, effective in inhibiting EDTF or its synthesis and which can be delivered across the blood-brain barrier.

A therapeutically effective amount of a compound of the present invention refers to an amount which is effective in controlling, or reducing Alzheimer's disease. The term “controlling” is intended to refer to all processes wherein there may be a slowing, interrupting, arresting, or stopping of the progression of the disease and does not necessarily indicate a total elimination of all disease symptoms.

The term “therapeutically effective amount” is further meant to define an amount resulting in the improvement of any parameters or clinical symptoms characteristic of Alzheimer's disease. The actual dose of the therapeutic agent will be different for the various specific molecules, and will vary with the patient's overall condition, the seriousness of the symptoms, and counterindications.

As used herein, the term “subject” or “patient” refers to a human who is afflicted with a particular Alzheimer's disease as indicated by senile dementia.

A therapeutically effective amount of the compound used in the treatment described herein can be readily determined by the attending diagnostician, as one skilled in the art, by the use of conventional techniques and by observing results obtained under analogous circumstances. In determining the therapeutically effective dose, a number of factors are considered by the attending diagnostician, including, but not limited to: the weight, age, and general health of the subject; the degree of or the severity of the disease; the response of the individual patient; the particular compound administered; the mode of administration; the bioavailability characteristic of the preparation administered; the dose regimen selected; the use of concomitant medication; and other relevant circumstances.

A therapeutically effective amount of the compositions of the present invention will generally contain sufficient active ingredient to deliver from about 0.1 μg/kg to about 50 mg/kg (weight of active ingredient/body weight of patient). Preferably, the composition will deliver at least 0.5 to 10 mg/kg, and more preferably at least 1 μg/kg to 1 mg/kg.

Practice of the method of the present invention comprises administering to a patient a therapeutically effective amount of the active ingredient(s), in any suitable systemic or local formulation, in an amount effective to deliver the dosages listed above to the cells which synthesize EDTF such as brain endothelial cell or cells which are affected by EDTF. The dosage can be administered on a regular schedule, for example, from one time per day.

Preferred amounts and modes of administration are able to be determined by one skilled in the art. One skilled in the art of preparing formulations can readily select the proper form and mode of administration depending upon the particular characteristics of the compound selected the disease state to be treated, the stage of the disease, and other relevant circumstances using formulation technology known in the art, described for example in Remington's Pharmaceutical Sciences, latest edition, Mack Publishing Co.

Pharmaceutical compositions can be manufactured utilizing techniques known in the art. Typically the therapeutically effective amount of the compound will be admixed with a pharmaceutically acceptable carrier.

Therapeutic agents contemplated herein may be administered by a variety of routes, for example, orally or parenterally (i.e. subcutaneously, intravenously, intramuscularly, intraperitoneally, or intratracheally).

For oral administration, the compounds can be formulated into solid or liquid preparations such as capsules, pills, tablets, lozenges, melts, powders, suspensions, or emulsions. Solid unit dosage forms can be capsules of the ordinary gelatin type containing for example, surfactants, lubricants and inert fillers such as lactose, sucrose, and cornstarch or they can be sustained release preparations.

In another embodiment, the compounds of this invention can be tabletted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the active ingredient in an aqueous or non-aqueous pharmaceutically acceptable solvent which may also contain suspending agents, sweetening agents, flavoring agents, and preservative agents as are known in the art.

For parenteral administration the compounds may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives, and buffers as are known in the art.

For surgical implantation, the active ingredients may be combined with any of the well-known biodegradable and bioerodible carriers, such as polylactic acid, hyaluronic acid and collagen formulations. Such materials may be in the form of solid implants, sutures, sponges, wound dressings, and the like. In any event, for local use of the materials, the active ingredients usually are present in the carrier or excipient in a weight ratio of from about 1:1000 to 1:20,000, but are not limited to ratios within this range. Preparation of compositions for local use are detailed in Remington's Pharmaceutical Sciences, latest edition, (Mack Publishing).

Additional pharmaceutical methods may be employed to control the duration of action. Controlled release preparations may be achieved through the use of polymers to complex or absorb the active ingredient. The controlled delivery may be achieved by selecting appropriate macromolecules (for example, polyesters, polyamino acids, polyvinyl, pyrrolidone, ethylenevinylacetate, methylcellulose, carboxymethylcellulose, or protamine, sulfate) and the appropriate concentration of macromolecules as well as the methods of incorporation, in order to control release.

Another possible method useful in controlling the duration of action by controlled release preparations is incorporation of the active agent into particles of a polymeric material such as polyesters, polyamino acids, polysaccharides, hydrogels, poly(lactic acid), or ethylene vinylacetate copolymers.

Alternatively, instead of incorporating the active agent into polymeric particles, it is possible to entrap these materials in microcapsules prepared, for example, by coacervation techniques or by interfacial polymerization (for example, hydroxymethylcellulose or gelatine-microcapsules and poly(methylmethacylate) microcapsules, respectively), in colloidal drug delivery systems (for example, liposomes, albumin microspheres, microemulsions, nano-particles, and nanocapsules), or in macroemulsions. Such techniques are disclosed in the latest edition of Remington's Pharmaceutical Sciences.

U.S. Pat. No. 4,789,734 describe methods for encapsulating biological materials in liposomes. Essentially, the material is dissolved in an aqueous solution, the appropriate phospholipids and lipids added, along with surfactants if required, and the material dialyzed or sonicated, as necessary. A good review of known methods is by G. Gregoriadis, Chapter 14. “Liposomes”, Drug Carriers in Biology and Medicine, pp. 287-341 (Academic Press, 1979). Microspheres formed of polymers or proteins are well known to those skilled in the art, and can be tailored for passage through the gastrointestinal tract directly into the blood stream. Alternatively, the agents can be incorporated and the microspheres, or composite of microspheres, implanted for slow release over a period of time, ranging from days to months. See, for example, U.S. Pat. Nos. 4,906,474, 4,925,673, and 3,625,214.

When the composition is to be used as an injectable material, it can be formulated into a conventional injectable carrier. Suitable carriers include biocompatible and pharmaceutically acceptable phosphate buffered saline solutions, which are preferably isotonic.

Where used herein, the term “EDTF” is intended to include the variants or analogues thereof. A “variant” of EDTF is meant to refer to naturally occurring molecules substantially similar to and having activity similar to EDTF^{BIM} or EDTF^{MV}. An “analogue” of EDTF is meant to refer to a synthetic version of EDTF substantially similar to a natural EDTF. A molecule is said to be “substantially similar” to another molecule if the sequence of amino acids in both molecules is substantially the same, and if both molecules possess a similar biological activity. Thus, provided that two molecules possess a similar activity, they are considered variants as that term is used herein even if one of the molecules contains additional amino acid residues not found in the other, or if the sequence of amino acid residues is not identical.

EDTF as disclosed herein is said to be “purified” or “substantially free of natural contaminants” if preparations which contain it are substantially free of materials with which this product is normally and naturally found.

For reconstitution of a lyophilized product in accordance with this invention, one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field.

The pharmaceutical composition may also be in the form of an aqueous solution containing many of the same substances as described above for the reconstitution of a lyophilized product.

The compounds can also be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

As mentioned above, the products of the invention may be incorporated into pharmaceutical preparations which may be used for therapeutic purposes. However, the term “pharmaceutical preparation” is intended in a broader sense herein to include preparations containing a protein composition in accordance with this invention, used not only for therapeutic purposes but also for reagent or diagnostic purposes as known in the art, or for tissue culture. The pharmaceutical preparation intended for therapeutic use should contain a “pharmaceutically acceptable” or “therapeutically effective amount” of an EDTF-inhibitor or antibody, i.e., that amount necessary for preventative or curative health measures. If the pharmaceutical preparation is to be employed as a reagent or diagnostic, then it should contain reagent or diagnostic amounts of an EDTF inhibitor or antibody.

Another therapeutic method contemplated herein comprises a method of treating a disease in a patient, one symptom of which is an abnormal level of endothelial-derived toxic factor, comprising exposing the patients serum to an anti-endothelial-derived toxic factor antibody to form an antibody: endothelial-derived toxic factor complex; and separating the serum from the antibody: endothelial-derived toxic factor complex and wherein said anti-endothelial-derived toxic factor antibody is a monoclonal antibody or antigen-binding fragment thereof that is reactive with endothelial-derived toxic factor and demonstrates an immunological binding characteristic of monoclonal antibody produced by at least one of hybridomas 2c2B, 2c2AA, 1d5A, 1d5D, and 2e4F.

Changes may be made in the formulation and the use of the various compounds and compositions described herein or in the methods or the steps or the sequence of steps of the methods described herein without departing from the spirit and scope of the invention as defined in the following claims.

What is claimed is:

1. A purified protein which can be isolated from brain microvessels of subjects having Alzheimer's disease, comprising:
incubating brain microvessels obtained from subjects having Alzheimer's disease in a culture medium under conditions wherein the medium is conditioned by the brain microvessels;
obtaining a quantity of conditioned medium from said brain microvessel culture;
exposing the quantity of conditioned medium to an affinity column having monoclonal antibody derived from at least one of the hybridomas selected from the group consisting of 2c2B, 2c2AA, 1d5A, 1d5D, and 2e4F or antibody which demonstrates an immunological binding characteristic of monoclonal antibody produced by at least one of hybridomas 2c2B, 2c2AA, 1d5A, 1d5D, and 2e4F; and
treating the affinity column to elute protein which is affinity bound to the antibodies thereon.

2. A purified protein which can be isolated from mammalian vascular endothelial cells by the method comprising:
culturing mammalian vascular endothelial cells;
treating said cultured endothelial cells to inhibit protein kinase C;
obtaining a quantity of conditioned medium from said treated cultured endothelial cells;
exposing the quantity of conditioned medium to an affinity column having monoclonal antibody derived from at least one of the hybridomas selected from the group consisting of 2c2B, 2c2AA, 1d5A, 1d5D, and 2e4F or antibody which demonstrates an immunological binding characteristic of monoclonal antibody produced by at least one of hybridomas 2c2B, 2c2AA, 1d5A, 1d5D, and 2e4F; and
treating the affinity column to elute protein which is affinity bound to the antibodies thereon.

3. A purified protein characterized by having:
high affinity binding for monoclonal antibody derived from at least one of the hybridomas of the group 2c2B, 2c2AA, 1d5A, 1d5D and 2e4F or antibody which demonstrates an immunological binding characteristic of monoclonal antibody produced by at least one of hybridomas 2c2B, 2c2AA, 1d5A, 1d5D, and 2e4F;
a molecular weight between 10 kDa and 50 kDa as determined by fractionation using Centricon devices; and
cytotoxicity specific for neuronal cells.

4. The protein of claim 3 wherein high levels of said protein evoke rapid death of neuronal cells via necrosis and wherein low levels of said protein evoke death of neuronal cells via apoptosis.

5. A method for detecting the presence of endothelial derived toxic factor in a sample, comprising:
incubating said sample with a monoclonal antibody or a specific polyclonal antibody which possesses specific binding for endothelial-derived toxic factor under conditions which provide for the formation of an endothelial-derived toxic factor-antibody complex; and
detecting the presence of said endothelial-derived toxic factor-antibody complex to determine whether endothelial-derived toxic factor is present in the sample.

6. The method of claim 5 wherein the monoclonal antibody is derived from at least one of hybridoma 2c2B, 2c2A, 1d5A, 1d5D and 2e4F or is antibody which demonstrates an immunological binding characteristic of monoclonal antibody produced by at least one of hybridomas 2c2B, 2c2AA, 1d5A, 1d5D, and 2e4F.

7. The method of claim 5 wherein the monoclonal or specific polyclonal antibody has an affinity for EDTF of K_{D}<10⁻⁸M.

8. A method of screening for compounds which inhibit the necrosis or apoptosis-inducing effects of EDTF on neuronal cells comprising:
providing a sample of neuronal cells;
treating the sample with a test compound;
exposing the treated sample to EDTF; and
examining the neuronal cells for evidence of inhibition or reduction of apoptosis or necrosis of the neuronal cells.

9. A method of screening for compounds which inhibit expression or activation of EDTF in microvascular endothelial cells comprising:
providing a sample of microvascular endothelial cells;
treating the sample with a test compound;
exposing the treated sample to a condition which normally induces the production of EDTF; and
examining the exposed sample for production of EDTF.

10. A method of screening for compounds which inhibit the necrosis or apoptosis-inducing effects of EDTF on neuronal cells comprising:
providing a sample of neuronal cells;
exposing the sample to EDTF;
treating the sample exposed to EDTF with a test compound; and
examining the neuronal cells for evidence of inhibition or reduction of apoptosis or necrosis of the neuronal cells.

11. A method of screening for compounds which inhibit expression or activation of EDTF in microvascular endothelial cells comprising:
providing a sample of microvascular endothelial cells;
exposing the sample to a condition which normally induces the production of EDTF;
treating the exposed sample with a test compound; and
examining the sample for production of EDTF.

12. A method of treating a disease in a patient, one symptom of which is an abnormal level of endothelial-derived toxic factor, comprising:
exposing the patient's serum to an anti-endothelial-derived toxic factor antibody to form an antibody:endothelial-derived toxic factor complex; and
separating the serum from the antibody:endothelial-derived toxic factor complex; and
wherein said anti-endothelial-derived toxic factor antibody is a monoclonal antibody or a specific polyclonal antibody or antigen-binding fragment thereof that is reactive with endothelial-derived toxic factor and demonstrates an immunological binding characteristic of monoclonal antibody produced by at least one of hybridomas 2c2B, 2c2AA, 1d5A, 1d5D, and 2e4F.
